(19) United States
(12) Patent Application Publication
(10) Pub. No.: US 2002/0173770 A1
(43) Pub. Date: Nov. 21, 2002

(54) Adhesive delivery system

(76) Inventors: Alan R. Flory, Golden Valley, MN (US); Scott P. Thome, St. Cloud, MN (US); Matthew F. Ogle, Oronoco, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 09/858,793

(22) Filed: May 16, 2001

(51) Int. Cl. A61M 25/16

(52) U.S. Cl. 604/537; 606/228; 606/214

(57) ABSTRACT

An adhesive delivery system described herein includes a reservoir of flowable adhesive and a delivery conduit with one or more apertures following a pattern. The one or more apertures of the delivery conduit preferably follow a curve. A prosthesis can be formed including an attachment surface and an adhesive delivery conduit associated with the attachment surface. The adhesive delivery conduit has at least one aperture and a central lumen connected to the aperture. The adhesive delivery systems are useful for the formation of medical devices, the implantation of medical devices and for wound healing.

### BACKGROUND OF THE INVENTION

The invention relates to tools for the delivery of a medical adhesive and methods involving the delivery of medical adhesives. The invention further relates to prostheses with adhesive delivery components attached to the prosthesis. The adhesive delivery systems are suitable to assist with the implantation of prostheses or for the manufacture of prostheses and other medical devices.

Medical glues/adhesives provide an alternative to suture, staples and the like for closing wounds in soft tissue, production of medical devices and implantation of medical devices, generally prostheses. Certain tissues, such as nerves and particular vital organs, are too delicate for suturing or stapling, so the use of surgical adhesives may be one of few viable repair options. Generally, the use of an adhesive for medical applications can be desirable due to potential sealing properties and uniform stress distribution.

Surgical adhesives, and medical adhesives generally, can be classified according to whether they include synthetic polymers, natural (biological) compositions or both. For example, a variety of synthetic urethane based polymers have been developed as surgical glues. The urethane based surgical adhesive compounds have been developed based on relatively low toxicity, strong binding and fast cure times. Natural surgical adhesives generally are based on proteins. For example, fibrin glues include the protein fibrinogen. Fibrinogen is used in natural wound healing mechanisms in humans and other mammals. Synthetic adhesives have the disadvantage of being potentially toxic. On the other hand, biological/natural adhesives generally have relatively low binding (cohesive) strengths and may have relatively rapid degradation times.

Medical adhesives can be used to attach various components of a medical device. The particular adhesive should be selected to be suitable for the particular materials to be secured. If the medical device is a prosthesis for implantation into a patient, generally the hardened form of the adhesive used in the manufacture of the prosthesis should itself be biocompatible, although adhesives can be used to produce non-implantable medical devices also.

Prostheses, i.e., prosthetic devices, are used to repair or replace damaged or diseased organs, tissues and other structures in humans and animals. Prostheses generally must be biocompatible since they are typically implanted for extended periods of time. For example, prostheses can include artificial hearts, artificial heart valves, ligament repair material, vessel repair, surgical patches constructed of mammalian tissue and the like.

While prostheses generally need to be constructed from biocompatible materials, a wide range of materials are suitable. Specifically, prostheses can be constructed from natural materials such as tissue, synthetic materials or a combination thereof. For example, prostheses formed from purely synthetic materials, such as mechanical heart valve prostheses, can be manufactured, for example, from biocompatible metals, ceramics, carbon materials, such as graphite, polymers, such as polyester, and combinations thereof.

Mechanical heart valves prostheses can be manufactured with rigid occluders or leaflets that pivot to open and close the valve. Alternatively, other heart valve prostheses can be constructed with flexible tissue leaflets or polymer leaflets. Prosthetic tissue heart valves can be derived from, for example, porcine heart valves or manufactured from other biological material, such as bovine pericardium.

A variety of approaches, such as suturing, stapling, clamping, adhering with adhesive and combinations thereof, can be used for the implantation of a prosthesis. Selection of a particular approach can depend on the particular implantation procedure. For example, attachment of a stentless aortic heart valve involves attachment on both inflow and outflow edges of the valve to secure the valve. Significant hemodynamic pressures are exerted against the valve in use. To maintain hemostasis under these conditions, it is necessary to secure the prosthetic valve along the outflow edge near the attached edge of the valve to distribute the load on the leaflets. Attachment along the outflow edge of the stentless valve is complex because the valve is within the aorta during the implantation.

### SUMMARY OF THE INVENTION

In a first aspect, the invention pertains to an adhesive delivery system comprising a delivery conduit with one or more apertures and a central lumen connecting with the one or more apertures. In some embodiments, the one or more apertures of the delivery conduit follow a pattern, such as a line, a curve or other extended pathway.

In another aspect, the invention pertains to a prosthesis comprising an attachment surface and an adhesive delivery conduit associated with the attachment surface. The adhesive delivery conduit has at least one aperture and a central lumen connected to the aperture.

In a further aspect, the invention pertains to a method for preparing a prosthesis for implantation, the prosthesis comprising an attachment surface, the method comprising placing a delivery conduit with at least one aperture along the attachment surface.

In addition, the invention pertains to a method for the implantation of a prosthesis comprising delivering a pattern of a composition selected from the group consisting of a medical adhesive, a medical adhesive component and a treatment compound through a delivery conduit comprising at least one aperture. In some embodiments, the delivery conduit delivers adhesive along a curve.

Furthermore, the invention pertains to a method of making a medical device, the method including applying an adhesive through a delivery conduit along a curve and forming an adhesive bond with the adhesive connecting to prosthesis components.

In further embodiments, the invention pertains to an adhesive delivery system comprising an adhesive delivery conduit comprising an resorbable polymer.

In other embodiments, the invention pertains to an adhesive delivery system comprising an adhesive delivery conduit having a plurality of microtubules extending from the surface of the adhesive delivery conduit.

### BRIEF DESCRIPTION OF THE DRAWINGS

FIG. 1 is a schematic perspective view of an adhesive delivery system.

FIG. 2 is a schematic perspective view of an alternative embodiment of an adhesive delivery system.

FIG. 3 is a side view of an embodiment of a delivery conduit with three slot openings.

FIG. 4 is a side view of an alternative embodiment of a delivery conduit with a single slit opening.

FIG. 5 is a perspective view of an adhesive delivery system with two reservoirs for adhesive components that deliver adhesive to a common conduit for delivery.

FIG. 6 is a perspective view of an adhesive delivery system with two adhesive reservoirs for delivery of adhesives or adhesive components into two parallel adhesive delivery conduits.

FIG. 7 is a perspective view of an embodiment of a delivery conduit along a closed curve.

FIG. 8 is a perspective view of an embodiment of a delivery conduit along an almost closed curve.

FIG. 9 is a perspective view of an embodiment of a delivery conduit along a non-planar closed curve.

FIG. 10 is a perspective view of a delivery conduit with two distinct perforated sections in fluid communication with each other.

FIG. 11 is a side view of a heart valve prosthesis with a delivery conduit attached near the scalloped outflow edge of the prosthesis.

FIG. 12 is a side view of a heart valve prosthesis with multiple delivery conduits attached along adjacent sections of the scalloped outflow edge and inflow edge of the prosthesis.

FIG. 13 is a side view of a heart valve prosthesis with a delivery conduit with perforated sections near both the inflow edge and the outflow edge of the prosthesis with the perforated sections being in fluid communication with each other.

FIG. 14 is a perspective view of an adhesive delivery conduit with microtentacles.

FIG. 15 is a sectional view of a bileaflet mechanical heart valve prosthesis, an adhesive delivery conduit for applying adhesive to an outer surface of an orifice ring forming the lumen of the valve, and a sewing cuff for application to the mechanical heart valve prosthesis following the application of adhesive, the cross section being taken through the center of the valve through both occluders.

FIG. 16 is a fragmentary sectional view showing the sewing cuff attached to the mechanical heart valve of FIG. 15.

FIG. 17 is a perspective view of an adhesive delivery conduit having a metal component and a flexible polymer component.

FIG. 18 is a top view of an annuloplasty ring.

### DETAILED DESCRIPTION OF THE ILLUSTRATIVE EMBODIMENTS

Improved adhesive delivery systems provide for the efficient and accurate delivery of adhesive for medical applications. In particular, the medical adhesives can be used for the implantation of prostheses, for the manufacture of medical devices and for the delivery of surgical adhesives for wound healing and the like. The improved adhesive delivery systems generally provide for the simultaneous or sequential delivery of adhesive along a perimeter or surface to facilitate proper and reproducible placement of the adhesive and to significantly improve efficiency and speed of adhesive placement for complex structures. When used in surgery, the adhesive delivery systems also can simplify and facilitate intricate implantation procedures and decrease corresponding costs. When used for manufacturing medical devices, improved uniformity can be achieved while simultaneously decreasing production time and decreasing costs through increased efficiency.

The improved medical adhesive delivery systems include a conduit such that adhesive is simultaneously or sequentially delivered along a pattern, such as a line, curve or other extended feature not necessarily localized at a point. The conduit can include one or more apertures, one or more slits or the like to achieve the desired adhesive delivery. Thus, with appropriate placement of the conduit, adhesive can be applied over an extended region without movement of the delivery system or portion thereof. The delivery conduit generally is attached to an adhesive reservoir. A valve, syringe or the like preferably controls the flow from the adhesive reservoir to the conduit such that flow can be initiated when the conduit is properly positioned.

In some preferred embodiments, the delivery conduit of the adhesive delivery system forms a desired pattern for adhesive delivery. A conduit with a selected structure can be used to apply adhesive along curves, generally closed curves or loops, either planar or non-planar. A closed curve is a curve that has no end points, such as a loop. Adhesive delivery systems having generally flexible structural components can be suitable to assist with the efficient implantation of prostheses with three dimensional shapes. The adhesive delivery systems can include components formed from self-expanding material, such as Nitinol® alloy, or from a material that has a set shape. Preferred prostheses involving biological conduits include, for example, vascular prostheses and cardiovascular prostheses, such as heart valves.

The adhesive delivery systems are particularly suitable for securing medical devices during implantation into the body. In general, relevant medical devices for implantation are prostheses that are formed to mimic a corresponding structure within the body. The prostheses can be used to replace or repair the corresponding native structure. Generally, prosthetic devices are suitable for long term implantation within a recipient patient. In preferred embodiments, the patient is an animal, preferably a mammal, such as a human.

In some embodiments, the adhesive delivery conduit is attached to the prosthesis for the delivery of the adhesive. Thus, the adhesive delivery conduit may function as a stent during the adhesive delivery process. When the adhesive delivery system and prostheses are properly positioned, the adhesive delivery system administers the adhesive at the appropriate location to attach the prosthesis to the patient. After delivering the adhesive, the conduit can be released from the prosthesis and removed from the patient or left within the patient following implantation of the prosthesis. If the conduit is left within the patient, it is preferably made from a material, such as a resorbable polymer, that is resorbed by the patient.

If the adhesive delivery conduit will be removed following adhesive delivery prior to the implantation of a prosthesis, the conduit may not be associated with the prosthesis during adhesive delivery. In these embodiments, the delivery conduit or an optional support structure supporting the delivery conduit preferably has a suitable shape such that the adhesive delivery system can be properly oriented prior to adhesive delivery. By using a separate structure, attachment of the delivery conduit to the prosthesis is avoided.

Suitable medical adhesives can include synthetic compounds, natural materials or a combination thereof. Suitable synthetic compound adhesives or components of a medical adhesive include, for example, cyanoacrylates and urethane polymers. Suitable natural material of a medical adhesive or components thereof include, for example, a variety of proteinaceous materials and associated binding agents. In certain embodiments, one or more components of the medical adhesive are a natural material, such as a protein, while one or more components are synthetic compounds, such as a crosslinking agent.

In certain embodiments, the medical adhesive is bioresorbable such that the adhesive is resorbed by the patient under natural physiological conditions after a suitable period of time. The period for resorption of the adhesive should be compatible with the time for the natural healing process. Generally, one or more components of the medical adhesive may be resorbable such that the adhesive is effectively absorbed by the patient over time. In embodiments involving the implantation of a prosthesis, natural healing processes eventually provide association of a substrate with natural tissue by way of extracellular structures that take the place of the adhesive. Once the adhesive is absorbed, any potential alterations of the mechanical properties of the tissue caused by the adhesive are replaced by more natural mechanical properties of healed natural tissue.

In some embodiments, the adhesives are multi-component adhesives. The adhesive delivery system can then be used to deliver a blend of the adhesive components or a subset of the adhesive components If only a fraction of the adhesive components are delivered with the adhesive delivery system, the remaining components are separately delivered. For example, the remaining components can be delivered separately, for example, by application over the surface of a medical device, by using a comparable adhesive delivery system to separately deliver the remaining adhesive components, by manually delivering the adhesive components or any other convenient approach. Once the remaining adhesive components are combined with the other adhesive components, the adhesive is complete and forms an adhesive bond upon solidification, i.e., hardening, or curing.

Similarly, additional reservoirs can be used to deliver other useful compositions for treatment of the prosthesis and or to facilitate the adhesive process. For example, an additional reservoir can be used for the application of treatment compounds, such as priming agents, cleaning solutions, growth factors, enzymes, detergents, salts, curing agents and the like. Some of these treatment compounds modify the surface while others remove cellular debris or the like. These treatment compounds can be added before, after or simultaneously with the adhesive or an adhesive compound. The order of addition of the treatment compound can be dictated in some circumstances by the function of the treatment compound.

The use of an adhesive to repair wounds in soft tissue is desirable due to its potential sealing properties and uniform stress distribution. The adhesive delivery systems described herein can facilitate the sealing of wounds, including complex wounds, by applying adhesive along a perimeter or surface of the wound. Thus, wound sealing can be simplified.

In addition, the adhesive delivery system can be used in the manufacture of various medical devices. The particular adhesive can be selected to be suitable for the particular materials to be attached. The adhesive delivery system is particularly suitable for the attachment of tubular components or other complex shapes, which can be formed from synthetic materials and/or natural materials, such as tissue. Adhesive bonds can be formed using the adhesive delivery systems to attach together medical device components with various shapes.

Alternatively, the medical adhesive can be used to attach a prosthetic device to the natural support tissue or other support structure within a patient. The adhesive delivery system is designed to apply the adhesive at appropriate locations to secure all or a portion of the prosthesis within the patient. The delivery conduit can be attached to the prosthesis for the adhesive delivery, or the adhesive delivery system can be used to apply the adhesive and then be removed prior to introduction of the prosthesis. If the adhesive delivery conduit is associated with the prosthesis, the delivery conduit can be removed after delivery of the adhesive, or the delivery conduit can be implanted into the patient along with the prosthesis if the conduit is sufficiently unobtrusive.

If the adhesive delivery system is used to deliver adhesive for the implantation of a prosthesis, a suitable adhesive is selected to bind to both the material of the prosthesis and the patient's natural tissue or other support structure. Once the prosthesis is ready to be secured to the native structure or other prosthetic components, a clamp or the like can be used to press or maintain the prosthesis against the native structure to allow the adhesive to form a seal. Use of an adhesive to implant a heart valve prosthesis may help to use a smaller sewing cuff or to eliminate the sewing cuff. The sewing cuff reduces the open lumen through the valve, so that reducing the size of the sewing cuff or eliminating the sewing cuff increases the open valve lumen and correspondingly improves valve performance. As another particular example, the adhesive delivery system can be used effectively to assist with the implantation of annuloplasty rings to support the annulus of damaged native heart valves.

Adhesive Delivery System

The adhesive delivery system includes a delivery conduit with a suitable aperture or apertures, in which the delivery conduit preferably is contoured to deliver adhesive along a predetermined pathway or pattern. The adhesive delivery system herein can include an adhesive reservoir. A suitable adhesive or adhesive component is located within the adhesive reservoir. Multiple adhesive reservoirs, each holding one or more adhesive components or adhesives, can be included if the adhesives or components are to be mixed during the adhesive delivery. The components can be mixed by generally simultaneous or serial delivery into a single passage, or can be applied separately.

The delivery conduit can be permanently attached to the adhesive reservoir or releasably attached to the adhesive reservoir. In addition, the delivery conduit can be attached to a medical device, such as a prosthesis. In preferred embodiments, the adhesive delivery system includes a suitable delivery or flow control component such that a desired amount of adhesive can be delivered through the delivery conduit.

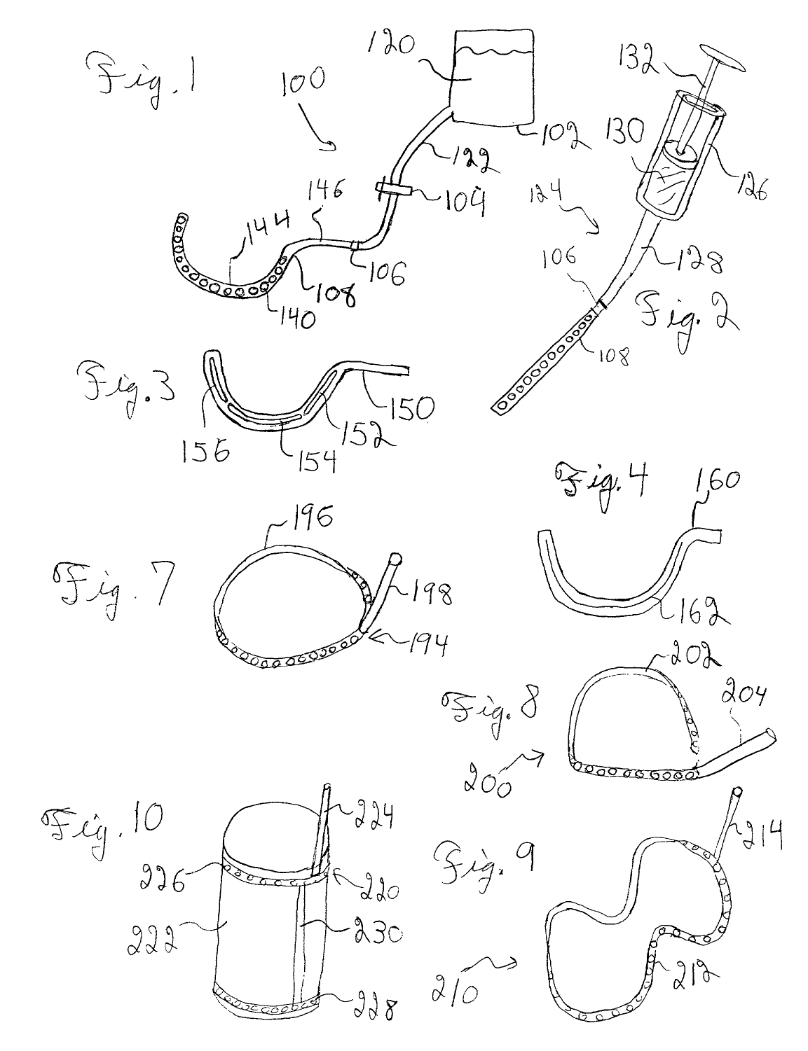

Referring to FIG. 1, adhesive delivery system 100 includes an adhesive reservoir 102, a flow control component 104, an optional releasable connection 106 and a delivery conduit 108. Adhesive reservoir 102 generally includes a supply of adhesive or adhesive components 120 and a tubular section 122 connecting to releasable connection 106 or directly to delivery conduit 108. The adhesive supply 120 may have properties that influence the design of the system. Specifically, adhesive 120 will have a particular viscosity and other flow characteristics that may influence the selection of flow control component 104, the dimensions of conduits 108, 122 and the size and shape of apertures in delivery conduit 108.

Adhesive reservoir 102 can be designed to hold sufficient adhesive 120 for a single application or for a plurality of applications. In particular, if the adhesive reservoir is disposable, the reservoir and associated components can be used once and discarded. Having a single use reservoir can prevent contamination of the adhesive supply 120. If the adhesive reservoir is only used once, a premeasured amount of adhesive 120 can be used such that the proper amount of adhesive is delivered when essentially all the adhesive 120 has been exhausted from the reservoir, accounting for any adhesive that remains in other portions of the delivery system. Alternatively, the adhesive reservoir can hold sufficient adhesive 120 for many applications, and other portions of the delivery system can be disconnected from the adhesive supply and discarded. Thus, various components of the adhesive supply system can be disposable after one use while other components can be reusable. In other embodiments, the entire adhesive delivery system is disposable after one use, or the entire adhesive delivery system is reusable.

In some embodiments, the adhesive reservoir is combined with the delivery conduit, a portion of the delivery conduit and/or a medical device associated with the adhesive delivery system. For example, the delivery conduit or a portion thereof can include the adhesive sealed within the delivery conduit under pressure. Thus, the adhesive is released by the over pressure when the seal is broken or removed. Similarly, the adhesive can be incorporated into the body of a prosthesis or other medical device, such that the medical device includes the adhesive reservoir. The adhesive reservoir within the medical device can include the delivery conduit, or it can be attached separately to a delivery conduit.

Flow control component 104 controls the flow of the adhesive between adhesive reservoir 102 and delivery conduit 108. Flow control component 104 can be a valve, a bulb, a seal, a syringe or the like. A plurality of flow control components can be used, if desired. The placement of the flow control component depends on the design of the flow control component. The selection and placement of the flow control component can also depend on the properties of the adhesive and/or the structure of the delivery conduit.

Flow control component 104 can be a valve or seal if surface tension and viscosity of the adhesive is not sufficient to prevent the flow through the aperture(s) of the delivery conduit 108. If a valve or seal is used, adhesive supply 120 can be open to the atmosphere or include a collapsible member such as a balloon, bag or the like to maintain flow without hinderance due to lack of driving pressure in the adhesive supply. Any reasonable valve design can be used, although a selected valve design to control flow preferably includes an easy to use controller, such as a lever or button. It is not generally desirable to have the adhesive supply open to the atmosphere since the adhesive can become contaminated, although appropriate filtration can be used to reduce the risk of contamination. If a seal is used to contain the flow, the seal can be broken or removed to initiate flow.

In alternative embodiments, flow control component 104 includes a syringe, a bulb or other compressible volume to direct flow from an adhesive reservoir to delivery conduit 108. These types of flow control devices can be used with adhesives having various viscosities, as long as the adhesive flows. For bulb based embodiments, squeezing the bulb forces adhesive from the reservoir to the delivery conduit and through apertures in delivery conduit 108.

Similarly, in preferred embodiments, a syringe can be used to deliver a measured amount of adhesive. Referring to FIG. 2, adhesive delivery system 124 includes a syringe 126 connected to delivery conduit 108 through tube 128 attached at connection 106. Syringe 126 includes an adhesive reservoir 130 and a plunger 132. Movement of plunger 132 controls the flow of adhesive or adhesive components from adhesive reservoir 130. Thus, pushing on plunger 132 decreases the volume of adhesive reservoir 130 and forces adhesive from the reservoir to the apertures in delivery conduit 108. By moving plunger 132, a selected amount of adhesive can be delivered. In some embodiments, syringe 132 includes markings to indicate particular volumes. Alternatively, the syringe can include a total volume to be delivered such that complete depression of plunger 132 results in the delivery of the desired amount of adhesive.

Commercial medical adhesive delivery devices can be adapted for flow control component 104. For example, CryoLife International, Inc., Kennesaw, Georgia, markets BioGlue® Surgical Adhesive in a prefilled sealed cartridge. The cartridge fits into a syringe device in which the plunger is depressed by squeezing a grip handle that fits within the palm of the user's hand. An applicator tip is attached to the cartridge to direct the adhesive through a narrow channel. To adapt the commercial system to the adhesive delivery device described herein, the applicator tip can be replaced with a conduit that connects between the adhesive cartridge and connector 106 or directly to delivery conduit 108.

Delivery conduit 108 provides advantages in the use of the adhesive delivery system. Specifically, the delivery conduit has a plurality of apertures or one or more extended apertures or slits such that the adhesive is delivered along one or more pathways or patterns. These patterns extend over a distance in contrast with a single point delivery. Adhesive delivery systems with these delivery conduits are in clear contrast with adhesive delivery systems that deliver adhesive through a small nozzle or tip that delivers adhesive at a single point. In preferred embodiments, the adhesive is delivered continuously or intermittently over a length that is generally at least about 0.5 cm, in other embodiments at least about 1 cm and in some preferred embodiments at least about 3 cm in length.

As shown in FIG. 1, delivery conduit 108 includes a plurality of apertures or holes 140. Apertures 140 can be placed close together so that adhesive flowing from the holes flows together to form an adhesive band that creates a seam when the adhesive bond forms upon hardening of the adhesive. Alternatively, the adhesive forms an intermittent arrangement along the pattern formed by delivery conduit 108 sufficient to form a desired adhesive bond. In other embodiments, holes 140 can be positioned to create other desired patterns of adhesive. Hole diameters can vary along the length of delivery conduit 108 to provide more or less adhesive delivery to a particular location. In addition, rather than a series of small holes, one or more extended apertures or slits can be used to deliver the adhesive over an extended area.

Delivery conduit 108 includes a perforated section 144 and a non-perforated extension 146. Non-perforated extension 146 is used to connect with flow control component 104. Non-perforated extension 146 can also be used as a grip to position perforated section 144 at the appropriate location. Delivery conduit 108 can also include additional handles or the like for convenient handling.

Referring to FIG. 3, an embodiment of a delivery conduit 150 is shown with three extended apertures 152, 154, 156. The number and dimensions of the extended apertures can be selected to yield the desired adhesive delivery over the area. Another alternative embodiment is shown in FIG. 4. In FIG. 4, delivery conduit 160 has a single elongated slit 162 for the continuous delivery of adhesive along a pathway corresponding to the slit.

The delivery conduit also provides an improved approach to the delivery of medical adhesives due to the shape of the delivery conduit. While the delivery conduit can be configured to deliver adhesive along a straight pattern, in some embodiments, the delivery conduit is curved such that adhesive is delivered along a curved pattern. The shape and size of the delivery conduit can be selected to conform to a feature of a medical device and/or anatomical features of a patient or other support structure. In particular, adhesive can be accurately delivered along a curve that becomes an adhesive bond between components within a medical device, between a medical device and anatomical structure of a patient or between two anatomical structures of the patient.

In some preferred embodiments, the delivery conduit forms a planar or non-planar curve, a closed curve or an open curve that approximates a closed curve. The pattern formed for adhesive delivery by aperture(s) in the delivery conduit may or may not be planar. The corresponding apertures of the delivery conduit similarly deliver adhesive along the corresponding closed curve. Flow of the adhesive after delivery can complete adhesive application along the closed curve if the delivery conduit only approximates a closed curve. Using delivery conduits that form a closed curve or approximately a closed curve, an adhesive seam can be formed connecting tubular-like structures and other complex shapes with inner and outer surfaces separated by an edge which itself forms a closed curve.

The adhesive delivery system can include a plurality of delivery conduits that are connected to one or more adhesive reservoirs, simultaneously or sequentially. For example, a plurality of curved delivery conduits can be combined to form an approximate closed curve for adhesive delivery. Similarly, a single delivery conduit can include a plurality of curved sections connected by one or more nonperforated tubular sections. Thus, a single delivery conduit can deliver adhesive simultaneously or sequentially to a plurality of curves at several separate sections of a medical device or on anatomical features of a patient.

In particularly preferred embodiments, the delivery conduit(s) forms a closed curve suitable for the delivery of adhesive for the implantation of vascular or cardiovascular prostheses. The closed curve formed by the delivery conduit(s) can be planar or nonplanar. For these embodiments, the closed curve can be circular, oval, elliptical, D-shaped, scalloped or other similar shapes. Similarly, delivery conduit(s) can include, for example, a scalloped section and a circular section to deliver adhesive to the inflow edge and the outflow edge of a vascular or cardiovascular prosthesis.

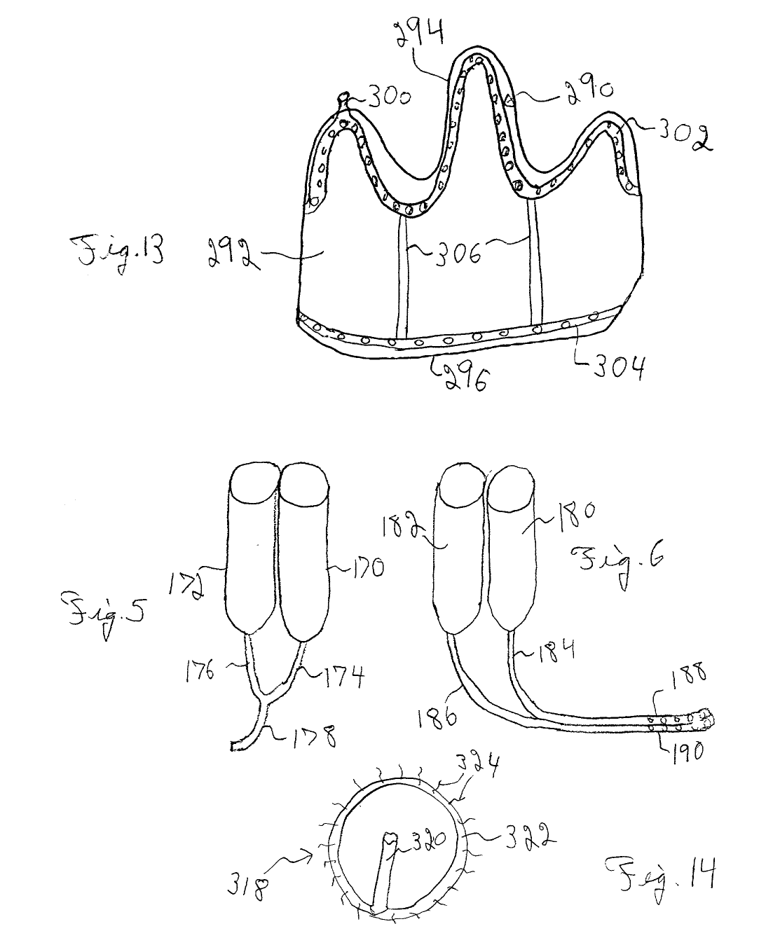

For multiple component adhesives, each component can be stored in separate reservoirs, i.e., two or more reservoirs. Conduits from separate reservoirs can lead to a common conduit for the mixing of the adhesive components, as shown in FIG. 5 for two adhesive components. In particular, adhesive components in reservoirs 170, 172 flows to conduits 174, 176 that combine in conduit 178. Similarly, two or more distinct adhesives in separate reservoirs can be mixed for improved performance of the resulting adhesive bond. Conduit 178 can be a portion of a delivery conduit or it can lead to a delivery conduit. The position of the mixing can be selected based on the degree of mixing desired and the hardening rate of the combined adhesive.

Alternatively, the separate reservoirs with different adhesive components can lead to parallel conduits that result in the mixing of the adhesive components upon delivery of the adhesive, as shown in FIG. 6 for two adhesive components. Specifically, reservoirs 180, 182 lead to conduits 184, 186. Conduits 184 and 186 lead to parallel adhesive delivery conduits 188, 190. The adhesive components mix following the delivery of the adhesive components from adhesive delivery conduits 188, 190. Similarly, two or more adhesives can be delivered in parallel to achieve binding with a combination of the properties of the multiple adhesives.

An embodiment of a delivery conduit forming a closed curve is shown in FIG. 7. In this embodiment, delivery conduit 194 includes a perforated planar closed curve or loop 196 having an oval or circular shape and a non-perforated tubular extension 198 with a lumen connected to the lumen of the perforated planar loop. Perforated planar loop 196 can be perforated around the entire circumference of the planar loop or over a portion of the circumference of the planar loop. In this embodiment, as in other embodiments herein, perforations, i.e., slits or holes, can be on one side, on opposite sides or other various configurations around perforated planar loop 196. Tubular extension 198 can be connected to a connector such as connector 106 of FIG. 1 for attachment to a flow control device and adhesive reservoir.

A variation on the embodiment of FIG. 7 is shown in FIG. 8. Delivery conduit 200 includes a perforated section 202 and non-perforated tubular extension 204. Perforated section 202 has a D-shape that almost, but not quite, forms a closed loop. The gap in the loop can be formed to effectively deliver the adhesive over a closed loop, either continuously or intermittently, without quite forming a structural closed loop with the perforated section 202. Alternatively, the gap can correspond to a gap in corresponding structure of the medical device.

An embodiment of a delivery conduit that delivers adhesive over a non-planar closed curve is shown in FIG. 9. Delivery conduit 210 includes a perforated section 212 and a non-perforated tubular extension 214. Perforated section 212 forms a non-planar closed loop such that adhesive is delivered through the perforations along a non-planar closed curve. Perforated section 212 can be configured to follow a stent or device.

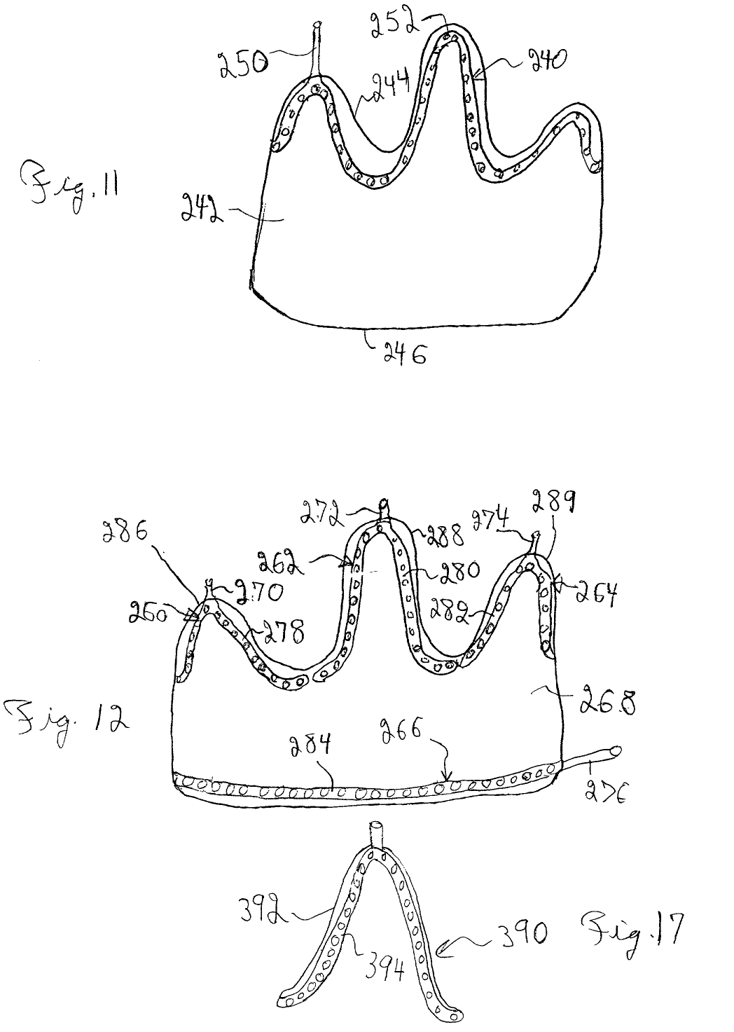

The delivery conduit can include distinct perforated sections for the delivery of adhesive along two or more pathways. Referring to FIG. 10, delivery conduit 220 is attached to a support frame 222 to facilitate positioning of delivery conduit 220. Support frame 222 also provides structural support to delivery conduit 220. While support frame 222 is shown as cylindrical, a variety of other shapes can be used, such as a tapered section, as appropriate for the particular application. Delivery conduit 220 includes a non-perforated tubular extension 224, a first perforated delivery section 226, a second perforated delivery section 228 and a non-perforated channel 230. Non-perforated channel 230 connects and provides for fluid flow between first perforated delivery section 226 and second perforated delivery section 228. Thus, adhesive flowing into non-perforated tubular extension 224 can flow into first perforated delivery section 226, non-perforated channel 230 and second perforated delivery section 228.

A delivery conduit 240 attached to a heart valve prosthesis 242 is shown in a side view in FIG. 11. Heart valve prosthesis 242 can be, for example, a stentless, aortic tissue heart valve prosthesis. During adhesive delivery for an aortic heart valve prosthesis, delivery conduit 240 is positioned between the prosthesis and the aorta and functions as a temporary or permanent stent. The valve prosthesis 242 has a scalloped outflow edge 244 and a generally circular inflow edge 246. The delivery conduit 240 includes a non-perforated tubular extension 250 and a scallop shaped perforated section 252 attached near outflow edge 244 that generally follows the shape of outflow edge 244. The positioning of perforated section 252 relative to the edge may depend on parameters, such as the viscosity of the adhesive, the dimensions of the delivery conduit and the implantation requirements of the device for proper function of the device. Thus, adhesive can be used to secure outflow edge 244 of the valve while suture or other fastener can be applied to secure the inflow edge.

An alternative embodiment is shown in FIG. 12 with four separate delivery conduits 260, 262, 264, 266 connected to heart valve prosthesis 268. Delivery conduits 260, 262, 264, 266 include non-perforated extensions 270, 272, 274, 276 and perforated sections 278, 280, 282, 284. Perforated sections 278, 280, 282 generally follow posts 286, 288, 289, respectively, of heart valve prosthesis 268. Perforated section 284 is located near the inflow edge of the valve. Two, three, five or other numbers of separate delivery conduits can similarly be used for adhesive delivery. As in FIG. 11, a delivery conduit may not be used at the inflow edge, or, alternatively, one or more delivery conduits could be used at the inflow edge and not at the outflow edge.

Another alternative embodiment is shown in FIG. 13 with delivery conduit 290 connected to heart valve prosthesis 292. Heart valve prosthesis 292 has a scalloped shaped outflow edge 294 and a generally circular inflow edge 296. The delivery conduit 290 includes a non-perforated tubular extension 300, a scallop shaped perforated section 302, a circular perforated section 304 and a plurality of non-perforated tubular connectors 306. Scalloped shaped perforated section 302 is attached near outflow edge 294. Circular perforated section 304 is attached near inflow edge 296. Non-perforated tubular connectors 306 provide an inflow path for medical adhesive from perforated section 302 to perforated section 304. Alternatively, all or some of tubular connectors 306 can be perforated for the application of adhesive along the side of the valve. Additional structural elements can be included to support the components of delivery conduit 290 that may or may not provide for flow of medical adhesive.

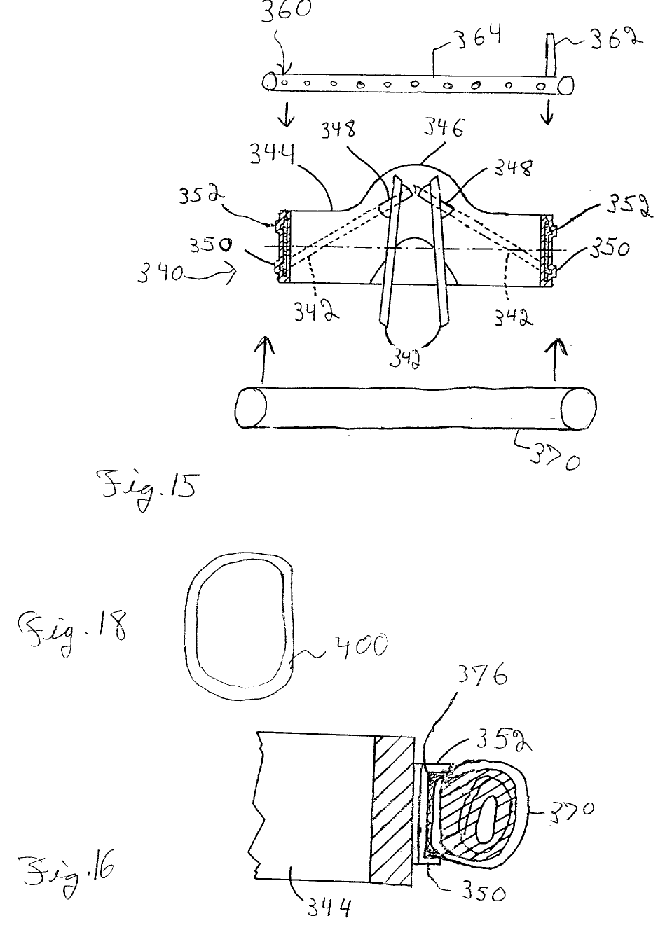

In alternative embodiments, the adhesive delivery conduit includes microtentacles/microtubules that extend outward from the surface of the conduit. The microtentacles/microtubules may or may not be fenestrated with pores at their ends and/or sides, in which the fenestrated embodiments are microtubules. A fragmentary view of an adhesive delivery conduit 318 with microtubules is shown in FIG. 14. A non-perforated conduit 320 is in fluid communication with a perforated section 322 of delivery conduit 318 having fenestrated microtentacles, i.e., microtubules 324. The pores of microtubules 324 function as the perforations of the conduit. The size of the microtubules can be selected to yield appropriate performance. Generally, the microtubules are less than about 30 millimeters (mm) in length, preferably from about 0.1 mm to about 5 mm and more preferably from about 1 mm to about 3 mm.

Microtentacles/microtubules can further function to grip the native tissue through mechanical interlocking to facilitate effective adhesive delivery. In these embodiments, a cuff may not be needed. The microtentacles/microtubules can be formed as part of a web or mesh that replaced the sewing cuff. Due to the added binding contributed by the microtentacles/microtubules, the mesh can be very thin. If the conduit is left in place following adhesive delivery, the prosthesis can be stabilized by the microtentacles/microtubules during implantation and hardening of the adhesive.

The components of the adhesive delivery system can be produced from any convenient non-toxic material that does not chemically react with the adhesive. Suitable materials include inert metals, such as stainless steel and Nitinol® (a nickel and titanium alloy), and inert polymers such as polyamides, i.e., Nylon®. Tubing for adhesive delivery can have a fixed lumen, or it can be expanding such that pressure from the flowing adhesive expands the tubing. In addition, a delivery conduit formed from flexible materials, such as Nitinol®, can expand outwardly in extent due to pressure from the fluid delivery such that the delivery conduit contacts the surface to which adhesive is applied. The adhesive delivery conduit can also be formed from resorbable polymers such that the delivery conduit can be left in the patient and is gradually degraded. Preferred resorbable polymers include, for example, resorbable polyesters, such as, for example, poly (hydroxy acids) and copolymers thereof, poly(ε-caprolactone), poly (dimethyl glycolic acid), and poly (hydroxy butyrate). Preferred resorbable polymers include, for example, D, L-polylactic acid (PLA), L-polylactic acid (PLA), poly(glycolic acid) (PGA), and copolymers of L-lactic acid, D-lactic acid and/or glycolic acid (PLA/PGA copolymers). Leaving the conduit within the patient simplifies the adhesive delivery process. In general, the cross sectional shape of the delivery components can be selected as desired unless there are structural constraints. In particular, channels and conduits for adhesive flow generally can have, for example, circular, oval, rectangular or similar cross sectional shapes.

Medical Adhesives

The adhesive delivery system described herein can be used for the delivery of any medical adhesive, although design modifications may be desirable depending on the adhesive properties. The medical adhesive, at least after solidifying, generally should be biocompatible, in that they are non-toxic, non-carcinogenic and do not induce hemolysis or an immunological response. The adhesive can be a single component adhesive or multi-component adhesive. Approaches for application of the adhesive described herein are designed to efficiently and accurately apply the adhesive and/or one or more of the adhesive components. If multi-component adhesives are used, the components can be mixed prior to application, during the application process, or only some of the adhesive components can be applied with the adhesive delivery system. Similarly, any catalysts and/or additives can be mixed with the adhesive prior to placement of the adhesive in the adhesive reservoir, during application, or after delivery of the adhesive. Suitable adhesives include synthetic adhesives, natural adhesives and combinations thereof.

With respect to synthetic adhesives, suitable one component adhesives include, for example, cyanoacrylate compounds. Particular cyanoacrylates include, for example, methyl cyanoacrylate, ethyl cyanoacrylate, n-propyl cyanoacrylate, isopropyl cyanoacrylate, n-butyl cyanoacrylate, isobutyl cyanoacrylate, n-amyl cyanoacrylate, isoamyl cyanoacrylate, 3-acetoxypropyl cyanoacrylate, 2-methoxypropyl cyanoacrylate, 3-chloropropyl cyanoacrylate, benzyl cyanoacrylate, phenyl cyanoacrylate, butyl-2-cyanoacrylate, fluorinated 2-cyanoacrylates and combinations thereof. Ethyl cyanoacrylate and butyl-2-cyanoacrylate are available from Loctite Corp., Hartford, Conn. These compounds harden quickly upon exposure to atmospheric humidity. The adhesive should be stored properly to avoid premature hardening.

Suitable two-component synthetic adhesives include, for example, urethane-based polymers, copolymers, and mixtures thereof. Polyurethanes are ester-amide derivatives of carboxylic acids. Urethane oligomers/prepolymers can be formed with terminal reactive functional groups. Because of the terminal functional groups, the prepolymers are particularly suitable for the formation of crosslinked mixed polymers exhibiting a range of desirable properties generally characteristic of polyurethanes and of the other components. With respect to the formation of an adhesive, in certain embodiments, the urethane prepolymer can be used as one component of the adhesive, with a crosslinking agent or agents being the other component or components of the adhesive.

Isocyanate (—NCO)—terminated urethane prepolymers are particularly suitable adhesive components. Polyurethanes including polyurethane prepolymers (urethane oligomers) can be formed either by the reaction of bischloroformates with diamines or the reaction of diisocyanates with polyhydroxy compounds. The approach to urethane polymerization involving diisocyanates with polyhydroxy compounds can be used to produce urethane prepolymers with isocyanate functional groups at their terminus. Suitable urethane prepolymers can be formed by the reaction of polyisocyanates with polyols.

Suitable polyisocynates include, for example, aromatic polyisocyanates containing 6-20 carbon atoms excluding the —NCO groups, such as o-, m- and p-phenylene diisocyanates (PDI), 2,4- and 2,6-tolulene d-socyanates (TDI), diphenylmethane-2,4′ and 4,4′-diisocyanates, diphenylmethane 2-4′- and 4,4′-diisocyanates (MDI), naphthalene-1,5-diisocyanate, triphenylmethane 4,4′,4″- trilsocyanate, polymethylenepolyphenylenepolyisocyanates (PAPI) obtained by phosgenation of aniline-formaldehyde condensation products, m- and p-isocyanato-phenyl sulfonyl isocyanate, and the like; aliphatic polyisocyanates containing 2-18 carbon atoms, such as ethylenediisocyanate; alicyclic polyisocyanates containing 4-15 carbon atoms, such as isophorone diisocyanate; araliphatic polyisocyanates containing 8-15 carbon atoms, such as xylylene diisocyanates; and modified polyisocyanates of these polyisocyanates, containing urethane, carbodiimide, allophanate, urea, biuret, urethdione, urethimine, isocyanurate and/or oxaolidong groups, such as urethane-modified TDI, carbodiimide-modified MDI, urethane modified MDI, and the like; as well as mixtures thereof.

For surgical adhesives, preferred polyisocyanates from this group include aromatic diisocyanates, particularly PDI, TDI (along with 2,4-and 2,6-isomers and mixtures of isomers with TDI), MDI (along with 4,4′- and 2,4′-isomers and mixtures isomers with MDI or PAPI), and modified polyisocyanates containing urethane, carbodiimide, allophanate, urea, biuret and/or isocyanurate groups, derived from PDI, TDI and/or MDI. Due to low toxicity, p-PDI (hereinafter PPDI) is particularly preferred. Alternative preferred embodiments include combinations of PPDI with a minor amount (usually up to about 500 by weight, preferably up to about 30% by weight) of one or more other polyisocyanates, such as aromatic polyisocyanates, particularly TDI, MDI, modified MDI and mixtures thereof.

Suitable polyols for the formation of the prepolymers include hydrophilic polyether polyols, other polyols and mixtures thereof. Representative suitable hydrophilic polyether polyols include adducts of ethylene oxide (hereinafter EO) or combinations of EO with other alkaline oxide(s) (hereinafter AO) formed with one or more compounds containing at least two active hydrogen atoms, such as polyhydric alcohols, polyhydric phenols, amines, polycarboxylic acids, phosphorous acids and the like. Suitable polyhydric alcohols include dihydric alcohols, such as ethylene glycol, trihydric alcohols, such as glycerol, and polyhydric alcohols having 4-8 or more hydroxyl groups, such as pentaerythritol. Representative suitable polyhydric phenols include mono- and poly-nuclear phenols, such as hydroquinone.

Suitable amines for the formation of polyether polyols include ammonia, alkanol amines, such as mono-, di- and tri-ethanol amines, aliphatic, aromatic, araliphatic and alicyclic monoamines, aliphatic, aromatic, araliphatic and alicylic polyamines, and heterocyclic polyamines.

For the formation of urethane prepolymers, addition of EO, or a combination of EO with an AO, to active hydrogen atom-containing compounds can be performed in conventional ways, with or without catalysts, such as alkaline catalysts, amine catalysts and acidic catalysts, under atmospheric pressure or at an elevated pressure, in a single step or in multiple steps. Addition of EO and AO may be performed by random-addition, block-addition or combination thereof, such as random-addition followed by block-addition. Random-addition is the preferred approach.

Preferred polyols for producing NCO-terminated urethane prepolymers have an average equivalent weight from about 100 to about 5,000, more preferably from about 200 to about 3,000 and generally 2-8 hydroxyl groups, preferably 2-4 hydroxyl groups.

The polyisocyanate and polyol preferably are mixed with a ratio of NCO/OH of about 1.5 to about 5.0 and more preferably from about 1.7 to about 3.0. The resulting prepolymers preferably have an NCO-content from about 1% to about 10% by weight and preferably about 2% to about 8% by weight. Lower NCO-contents can result in a low binding strength and higher NCO-contents can lead to brittle bonds.

The polyisocyanates and polyols react to form urethane prepolymers. These prepolymers are moderate molecular weight oligomers. The size of the oligomers is controlled by the relative amounts of NCO functional groups and OH functional groups. Since the NCO functional groups are added in excess, the polymerization terminates when all of the OH groups have reacted. The unreacted NCO groups form the basis for further polymerization to form the final adhesive. Bioresorbable urethane based adhesives can be made from suitable hydrophilic urethane prepolymers.

Suitable compositions for the second component of the urethane based medical adhesives include polyols, such as the polyols used to form the prepolymer. The amount of polyols added can be based on the number of functional groups remaining unreacted in the urethane prepolymer. Alternatively, the second component of the urethane oligomer adhesive can be an unsaturated cyano compound containing a cyano group attached to a carbon atom involved in the polymerizable double bond, such as cyano acrylic acids and esters thereof. Examples of these unsaturated cyano compounds include, for example, cyanoacrylic acid, cyano methacrylic acid, methyl cyanoacrylic acid, methyl cyanomethacrylic acid, ethyl cyanoacrylic acid, ethyl cyanomethacrylic acid, isobutyl cyanoacrylic acid, isobutyl cyanomethacrylic acid, corresponding esters, acrylonitriles, methacrylonitriles, cyanoacrylonitriles, cyanomethacrylonitriles and mixtures thereof. Such adhesives are described in U.S. Pat. No. 4,740,534 to Matsuda et al., incorporated herein by reference. Mixtures of polyols and unsaturated cyano compounds can be used as the second or additional component(s) of the adhesive.

The urethane based adhesive composition generally comprises about 20 to about 90 percent by weight urethane prepolymer and preferably about 30 to about 70 percent by weight urethane prepolymer. The ratio of urethane prepolymer to unsaturated cyano compound can be varied to achieve a desired flexibility. The use of a higher percentage of urethane prepolymer results in an adhesive with greater flexibility. A catalyst can be added if desired. Urethane based medical adhesives are discussed further in published PCT application WO00/43050, entitled “Medical Adhesives,” incorporated herein by reference.

Adhesives based on components that are natural compositions generally are based on inherent natural binding affinities and corresponding biological responses. Generally, one or more components of the adhesive is a protein or protein based compound. Protein is intended to be interpreted broadly in terms of any compound with a polypeptide (i.e., amino acid) component, and may include derivatives of natural proteins and polypeptides with additional covalently or non-covalently attached components, such as additional polypeptides, nucleotides, carbohydrates, and other organic or inorganic compounds. Protein components generally contain amino acids with side chains with functional groups useful for binding with the remaining adhesive components. Also, if the substrate is a crosslinked tissue, an adhesive component can replace functional groups that had been eliminated in the tissue substrate by reactions during the crosslinking process.

A type of biological adhesive is based on the protein fibrinogen. Fibrinogen, also known as factor I, is involved in natural blood clotting processes The protein thrombin removes one or two peptides from fibrinogen to form fibrin. Thrombin is also involved in the blood clotting process. A variety of fibrin adhesives have been based on the crosslinking of fibrin. Fibrin glues generally involve combinations of fibrinogen, thrombin and Factor XIII. Factor XIII also is involved in the natural wound healing mechanism. Factor XIII, also known as fibrin stabilizing factor, is activated by thrombin, and converts soluble fibrin to an insoluble clot. Fibrin adhesives polymerize and also covalently crosslink with collagen and other tissue components to form a liquid tight bond. The final amounts of the fibrinogen, thrombin or factor XIII components in the complete adhesive can be adjusted, as desired, to yield selected adhesive properties, such as strength and/or cure times, or for convenient application.

U.S. Pat. No. 4,818,291 to Iwatsuki et al., incorporated herein by reference, describes the inclusion of silk-fibroin protein into a fibrin glue to enhance its mechanical strength. Fibrin adhesives may also contain albumin, as described in U.S. Pat. No. 4,414,976 to Schwarz et al., incorporated herein by reference.

Another type of adhesive includes a biological component and a synthetic component. Generally, the biological component includes a protein. For example, gelatin-resorcinol aldehyde adhesives involve a gelatin-resorcinol material that is formed by heating gelatin and resorcinol. Gelatin is formed by hydrolytic activity on collagen protein. Formaldehyde, glutaraldehyde or the like can be used to crosslink the gelatin-resorcinol material to complete the formation of the glue.

A similar adhesive is formed from water soluble proteinaceous material and di- or polyaldehydes. The proteinaceous materials may be purified proteins or mixtures of proteins. Preferred proteins include albumins, including ovalbumins. Particularly preferred proteins include serum albumins of human or animal origin. Suitable water soluble di- or polyaldehydes include glyoxal and glutaraldehyde. The adhesive cures within a minute or less after the application of the aldehyde by spraying a layer over a coating of the proteinaceous material. Such adhesives are described further in U.S. Pat. No. 5,385,606 to Kowanko, incorporated herein by reference.

Similar adhesives based on proteinaceous material have been described in U.S. Pat. No. 5,583,114 to Barrows et al., incorporated herein by reference. Again, the proteinaceous material preferably includes serum albumin as a primary component. The second component includes bifunctional crosslinking agents, with preferred crosslinking agents including polyethylene glycol with a molecular weight ranging from about 1,000 to about 15,000. The polyethylene glycol can be modified to incorporate leaving groups to activate the crosslinking agent to bind at primary or secondary amines of the proteins. Suitable leaving groups include, for example, succinimidyl, maleimidyl, phthamimidyl, other imides, heterocyclic leaving groups such as imidazolyl, aromatic leaving groups such as nitrophenyl, and fluorinated alkylsulfone leaving groups such as tresyl (CF₃—CH₂SO₂—O—). A linking group can be bonded between the polyethylene glycol and the leaving group.

The adhesives can contain additives to modify the mechanical properties of the adhesive. Suitable additives include, for example, fillers, softening agents and stabilizers. Representative fillers include, for example, carbon black and metal oxides, silicates, acrylic resin powder, and various ceramic powders. Representative softening agents include, for example, dibutyl phosphate, dioctylphosphate, tricresylphosphate, tributoxyethyl phosphates and other esters. Representative stabilizers for the urethane based polymers include, for example, trimethyldihydroquinone, phenyl-β-naphthyl amine, p-isopropoxydiphenylamine, diphenyl-p-phenylene diamine, and the like. The protein based adhesives can also contain sugars such as glucose or sucrose to improve solubility, and stabilizers, including heparin. Fibrin glues can contain additional components, such as an inhibitor of fibrinolysis (anti-fibrolytic agents), for example, aprotinin and/or transexamic acid, with calcium chloride.

The properties of the adhesive generally are selected based on the particular application. In particular, the hardening rate and the adhesive strength can be selected based on the discussion above by a person of skill in the art.

Medical Devices

Preferred medical devices include all medical devices that contact body fluids and/or tissue. These articles can be organized generally into three groups: implanted devices, percutaneous devices and cutaneous devices. Implanted devices broadly include articles that are fully implanted in a patient, i.e., are completely internal. Percutaneous devices include items that penetrate the skin, thereby extending from outside the body into the body. Cutaneous devices are used superficially, for example, at a moist membrane, such as within a patient's mouth.

Implanted devices and components thereof include, without limitation, prostheses such as artificial heart valves, heart valve stents, heart valve leaflets, orifice rings of mechanical heart valves, pacemakers, electrical leads such as pacing leads, defibrillators, artificial organs such as artificial hearts, ventricular assist devices, anatomical reconstruction prostheses such as jaw implants, pericardial patches, surgical patches, coronary stents, vascular grafts, vascular, cardiovascular and structural stents, vascular and cardiovascular shunts, biological conduits, pledgets, suture, annuloplasty rings, stents, staples, connectors, valved grafts, dermal grafts for wound healing, orthopedic and spinal implants, orthopedic pins, intrauterine devices (IUDs), urinary stents, permanently indwelling pericardial devices, nerve conduits, neurological devices, maxial facial reconstruction plating, dental implants, intraocular lenses, clips, sternal wires, bone prostheses, skin prostheses, ligament prostheses, tendon prostheses, and combinations thereof.

Percutaneous devices include, without limitation, angioplasty balloons, catheters of various types, cannulas, drainage tubes such as chest tubes, surgical instruments such as forceps, retractors, needles, and gloves, and catheter cuffs. Catheters can be used for accessing various bodily systems such as the vascular system, the gastrointestinal tract, or the urinary system.

Cutaneous devices include, without limitation, burn dressings, wound dressings and dental hardware, such as bridge supports and bracing components. These biocompatible articles can be made from the biocompatible materials described below.

While medical adhesives can be used in any of the medical devices described above, a few medical devices are of particular interest. Such devices of particular interest include, for example, heart valve prostheses and annuloplasty rings. In particular, the adhesive delivery devices can be used in the implantation of stentless aortic prostheses and annuloplasty rings. Suitable delivery conduits for the implantation of heart valve prostheses are shown, for example, in FIGS. 11-13, and suitable delivery conduits for the implantation of annuloplasty rings are shown, for example, in FIGS. 7 and 8.

Biocompatible Materials

Preferred medical devices, which are designed to contact the body fluids or tissues of a patient, generally include biocompatible materials. Appropriate biocompatible materials can be formed from natural materials, synthetic materials or combinations thereof. Suitable biocompatible materials include, for example, tissue, polymers, metal, carbon materials, and ceramics.

Natural tissues may be obtained from, for example, native heart valves, portions of native heart valves such as aortic roots, walls and leaflets/cusps, pericardial tissues, such as pericardial patches, connective tissues, bypass grafts, tendons, ligaments, skin patches, blood vessels, cartilage, dura mater, skin, bone, fascia, submucosa, umbilical tissues, and the like. Natural, i.e., biological, material for use in the invention includes relatively intact living tissue, decellularized tissue and recellularized tissue.

Natural tissues are derived from a selected animal species, typically mammalian, such as human, bovine, porcine, seal, equine or kangaroo. These natural tissues generally include collagen-containing material. Natural tissue is typically, but not necessarily, soft tissue. Appropriate tissues also include tissue equivalents such as tissue-engineered material involving a cell-repopulated matrix, which can be formed from a polymer or from a natural tissue. Tissue materials are particularly useful for the formation of tissue heart valve prostheses.

Tissues can be fixed by crosslinking. Fixation provides mechanical stabilization, for example, by preventing enzymatic degradation of the tissue. Glutaraldehyde or formaldehyde is typically used for fixation, but other fixatives can be used, such as other polyfunctional aldehydes, epoxides, and genipin and derivatives thereof. Tissues can be used in either crosslinked or uncrosslinked form, depending on the type of tissue, the use and other factors. Generally, if xenograft tissue is used, the tissue is crosslinked and/or decellularized.

Relevant synthetic materials include, for example, polymers, ceramics and metals. Appropriate ceramics include, without limitation, hydroxyapatite, alumina and pyrolytic carbon. Appropriate metals include, for example, titanium, cobalt, stainless steel, nickel, iron alloys, cobalt alloys, such as Elgiloy®, a cobalt-chromium-nickel alloy, and MP35N, a nickel-cobalt-chromium-molybdenum alloy, and Nitinol®, a nickel-titanium alloy. Appropriate synthetic materials include hydrogels and other synthetic materials that cannot withstand severe dehydration.

Biocompatible materials can be fabricated from synthetic polymers as well as purified biological polymers. These synthetic polymeric materials can be formed into fibers and then can be woven or knitted into a mesh to form a matrix or similar structure. Alternatively, the synthetic polymer materials can be molded or cast into appropriate forms.

Appropriate synthetic polymers include, without limitation, polyamides (e.g., nylon), polyesters, polystyrenes, polyacrylates, vinyl polymers (e.g., polyethylene, polytetrafluoroethylene or other halogenated polymers such as polyvinylchloride, polypropylene, other polyolefins, ethylene-propylene copolymers, and ethylene-propylene-diene monomer copolymers (EPDM)), polycarbonates, polyacetals (e.g., Delrin®), polyurethanes, polydimethyl siloxanes, cellulose acetates, polymethylmethacrylates, ethylene vinyl acetates, polysulfones, nitrocelluloses, polyetheretherketones (PEEK) and copolymers and mixtures thereof. Based on desirable properties and experience in the medical device field, preferred polymers include, for example, polyetheretherketones, polyacetals, polyamides, polyurethanes, polytetrafluoroethylenes, polyester teraphthalates, polycarbonates, polysulfones, polypropylenes, and copolymers and mixtures thereof.

Biological polymers can be naturally occurring or produced in vitro by, for example, fermentation and the like. Purified biological polymers can be appropriately formed into a substrate by techniques such as weaving, knitting, casting, molding, extrusion, cellular alignment and magnetic alignment. Suitable biological polymers include, without limitation, collagen, elastin, silk, keratin, gelatin, polyamino acids, polysaccharides (e.g., cellulose and starch) and copolymers thereof.

Assembly of Medical Devices

The adhesive delivery systems described herein can be used for the assembly of medical devices. The application of the adhesive with the adhesive delivery system can be performed by automated equipment, by hand with appropriately skilled operators or by a combination thereof. In any case, the use of the adhesive delivery systems described herein facilitate adhesive delivery, improve reproducibility and accuracy of adhesive delivery.

The adhesive delivery systems are particularly advantageous for the rapid delivery of adhesive along curved surfaces. Delivery of adhesive along curved surfaces is especially useful for medical devices with three dimensional shapes. If the delivery conduit is positioned by hand, the adhesive delivery conduits provide for relatively easy positioning of the components needed to apply the adhesive along curved surfaces. Automated/robotic actuators can be used to position the adhesive delivery conduit at an appropriate position relative to a component of a medical device.

The adhesive delivery systems are particularly suitable for the assembly of heart valve prostheses. For example, sewing cuffs, fabric coverings or the like can be secured with adhesive applied along a circular or other curved surface. Similarly, stents and other support structures can be secured with adhesive to form attached edges of flexible leaflets. Flexible leaflets can be formed from polymers, such as polyurethane, or tissue. The stents generally have a closed, non-planar scalloped shape along a tubular construct. Alternatively, stents can be applied temporarily to facilitate the adhesive application process and, then, be removed to form a stentless prosthesis. This curved scalloped shape is amenable to efficient application of adhesive using the adhesive delivery systems.

As an example of medical device component assembly with adhesive, mechanical heart valve 340 with two leaflets or occluders 342 is shown in FIG. 15. Mechanical heart valve 340 includes a ring 344 that forms the open lumen of the valve. Ring 344 includes extensions 346 that have recesses 348 in which the occluders 342 pivot. The closed position of occluders 342 is shown with phantom lines. Two flanges 350, 352 extend around the circumference of ring 344. A sewing cuff can be secured within the groove or space formed between flanges 350, 352.

An adhesive delivery conduit 360 is shown positioned above mechanical heart valve 340. Adhesive delivery conduit 360 includes a non-perforated extension 362 for connecting to an adhesive reservoir and a perforated ring 364. Perforated ring 364 has a diameter such that it can be lowered or positioned into place between flanges 350, 352. Arrows indicate the motion of perforated ring 364 for positioning along mechanical valve 340. Perforated ring 364 has perforations located along the inner diameter. Perforated ring 364 can be slightly elastic such that some force can be required to clear flange 352. Similarly, perforated ring 364 can be an open curve approximating a closed curve, to facilitate positioning of ring 364 for adhesive delivery.

A sewing cuff 370 is shown positioned below mechanical heart valve 340. Sewing cuff 370 has a suitable diameter for placement along ring 344 between flanges 350, 352. Sewing cuff 370 generally is elastic or flexible for placement between flanges 350, 352.

To attach sewing cuff 370 to mechanical heart valve 340, adhesive delivery conduit 360 is placed with perforated ring 364 between flanges 350, 352. Adhesive is deposited in the groove between flanges 350, 352, and adhesive delivery conduit 364 is removed. Then, sewing cuff 370 is placed between flanges 350, 352. When the adhesive hardens, the sewing cuff 370 is secured to mechanical heart valve prosthesis 340. The attached sewing cuff 370 is shown in the fragmentary view of FIG. 16. Adhesive 376 holds sewing cuff 370 between flanges 350, 352. In alternative embodiments, the adhesive is applied to the sewing cuff before placement between flanges 350, 352.

Prosthesis Implantation

The adhesive delivery systems can be used advantageously for the surgical implantation of prostheses, including implanted repair devices, such as annuloplasty rings. In some embodiments, the delivery conduit is attached to the prosthesis for the delivery of the adhesive, or the delivery conduit is attached to a support structure mimicking the structure of the prosthesis to provide for proper alignment.

It can be advantageous to attach the delivery conduit to the prosthesis such that the proper alignment of the prosthesis correspondingly aligns the delivery conduit for the delivery of the adhesive along the desired tissue in the patient. Thus, adhesive can be properly applied along curved surfaces quickly and accurately just by visually aligning the prosthesis prior to initiating adhesive delivery. If desired, the delivery conduit can be removed from the prosthesis. For example, if the delivery conduit is attached to the prosthesis with suture, the delivery conduit can be removed by cutting the suture and separating the conduit from the prosthesis. Other fasteners, such as hooks or clips, can be used to releasably attach the delivery conduit to the prosthesis. Then, the implantation of the prosthesis can be completed without the presence of the delivery conduit.

In alternative embodiments, the delivery conduit is left attached to the prosthesis following adhesive delivery. In these embodiments, the delivery conduit should be nonobtrusive, such that the delivery conduit does not interfere with the function of the prosthesis. The delivery conduit generally is released from the adhesive reservoir and other components of the adhesive delivery system. The delivery conduit left on the prosthesis can be resorbable.

Similarly, a portion of the delivery conduit can be left associated with the prosthesis while other portions are removed. For example, an resorbable, adhesive delivery conduit can be connected to a Nitinol® alloy support to provide a shape to the composite structure. The Nitinol® component is removed and the resorbable portion remains associated with the prosthesis. An adhesive delivery conduit 390 with a Nitinol® support component 392 and an resorbable perforated section 394 is shown in FIG. 17. Adhesive delivery conduit 390 can be substituted for one of the adhesive delivery conduits in the adhesive delivery system shown in FIG. 12.

In other embodiments, the delivery conduit is secured to a structure approximating the overall prosthesis shape, or the delivery conduit itself can be shaped sufficiently similar to the prosthesis to provide for proper alignment of the delivery conduit. The delivery conduit and any corresponding support structure can be positioned like the prosthesis for the application of the adhesive. Following the delivery of the adhesive, the delivery conduit and support structure are removed and the prosthesis is positioned for attachment.

In particular, the adhesive delivery systems are suitable for the implantation of tubular prostheses, such as vascular and cardiovascular prostheses. Tubular prostheses generally must be attached along curved surfaces to form a fluid tight seal. Adhesives discussed above are suitable to from these seals if they are appropriately applied along the curved surface, generally along a closed curve or an approximate closed curved. For example, blood vessel prostheses, with or without valves, can be secured to a native blood vessel using adhesive applied along a circular or a more complex closed curve at each end of the vascular prosthesis. In addition, adhesive can be applied using a delivery conduit along a generally linear pattern along the length of the delivery conduit.

Similarly, the adhesive delivery system can be used for the implantation of heart valve prostheses. The adhesive delivery system is particularly suitable for the implantation of stentless aortic heart valves. A stentless aortic heart valve is secured along a scalloped edge on the outflow side of the valve. Since the valve is within the aorta during the implantation, the attachment of the outflow edge with suture is challenging and time consuming. Adhesive can be delivered along the scalloped outflow edge of the prosthesis to secure this edge of the prosthesis. Precise positioning of the adhesive provides corresponding precise positioning of the prosthesis. Thus, for example, an aortic heart valve prosthesis can be accurately positioned to clear and avoid the ostia. The circular shaped inflow edge can be secured with adhesive and/or other fasteners, such as suture or staples. In addition, a mechanical heart valve prosthesis, as in FIG. 15 can be implanted by the delivery of adhesive to the cuff of the valve.

While the adhesives are generally selected to provide required binding strength, additional fasteners, such as suture, staples, clamps and the like, can be used to supplement the adhesive or to secure the prosthesis while the adhesive is solidifying. If the fasteners are used for temporarily securing the prosthesis while the adhesive is solidifying, the fasteners may be removed after hardening of the adhesive. Generally, any fastener used to facilitate adhesive attachment is used to a significantly lesser degree than if the fastener was used as the only securing tool. For example, a few stay sutures can be applied along the outflow edge of a stentless aortic heart valve prosthesis to hold the valve in place while an adhesive is solidifying. The application of these few stay sutures requires much less effort and time than the complete suturing of the outflow edge of the prosthesis.

In other embodiments, the prosthesis is manipulated following application of the adhesives prior to hardening of the adhesive. For example, if adhesive is applied to facilitate the implantation of an annuloplasty ring, the annuloplasty ring can be shaped and/or oriented following application of the adhesive. Specifically, for use in mitral valve repair, an annuloplasty ring 400 can be shaped to have a D-shape, as shown in FIG. 18. A holder can be used to hold and shape the ring for an appropriate amount of time while the adhesive begins to set.

Use of the adhesive delivery system provides for more efficient and quicker implantation of prostheses to speed up the surgical process and reduce time in surgery. In addition, the adhesive delivery system provides more reproducible results and uniformity of the sealing of the prosthesis for improved results of the implantation. Controlled delivery is safer for the patient since the chance of improper adhesive delivery is reduced and consistency of the results provides for improved long term performance and decreased reoperation rates.

Storage, Delivery and Use

In general, the adhesive is stored in an air tight container prior to use to prevent evaporation of solvent and/or contact with oxygen. If the adhesive has multiple components, these can be separately stored or stored in combined form if the combination of the components does not lead to premature hardening of the adhesive. Components of the adhesive delivery system that, in use, contact the patient or the medical device preferably are stored in a sterile container, such as a sealed plastic bag. However, components that are sterilized for reuse can be stored in nonsterile conditions on the assumption that they will be sterilized prior to use.

If the adhesive delivery system or a component thereof, such as the delivery conduit, is connected to a prosthesis for adhesive delivery, special storage considerations may be required. For example, if a delivery conduit is attached to a tissue-based prosthesis, the prosthesis with the delivery conduit generally would be stored under moist conditions to prevent dehydration of the tissue. For example, the prosthesis and delivery conduit can be stored submerged in a sterilizing fluid, such as an aqueous alcohol solution or an aqueous glutaraldehyde solution.

The components of the adhesive delivery system can be packaged separately. Desired packaging configurations may depend on the eventual use of the adhesive delivery system. Similarly, if the adhesive delivery system is used in association with a prosthesis, the prosthesis and the adhesive delivery system or components thereof can be associated with each other prior to packaging, or the prosthesis and adhesive delivery system and/or components thereof can be packaged separately. The prosthesis and adhesive delivery system can be associated with each other prior to use if they are not assembled together prior to packaging. For example, a couple of stay sutures can be used to connect an adhesive delivery conduit with a tissue-based prostheses at a medical facility or a manufacturing facility. Similar fastening approaches can be similarly used as appropriate for the particular materials.

With the adhesive delivery system or components thereof appropriately stored, the adhesive delivery system or component can be shipped to a manufacturing facility or to medical personnel. Generally, the adhesive delivery system is packaged along with appropriate instructions and other packaging information. If the adhesive delivery system includes both disposable and nondisposible components, the reusable components and disposable components are generally packaged and shipped separately.

The embodiments described above are intended to illustrative and not limiting. Additional embodiments are within the claims. Although the present invention has been described with reference to preferred embodiments, workers skilled in the art will recognize that changes may be made in form and detail without departing from the spirit and scope of the invention.

What is claimed is:

1. An adhesive delivery system comprising a delivery conduit with one or more apertures and a central lumen connecting with the one or more apertures, wherein the one or more apertures follow a pattern.

2. The adhesive delivery system of claim 1 wherein the one or more apertures follow a curve.

3. The adhesive delivery system of claim 2 wherein the curve is a closed curve.

4. The adhesive delivery system of claim 2 wherein the curve is planar.

5. The adhesive delivery system of claim 2 wherein the curve is non-planar.

6. The adhesive delivery system of claim 1 wherein the one or more apertures comprises a plurality of apertures.

7. The adhesive delivery system of claim 6 wherein the plurality of apertures comprise a circular aperture.

8. The adhesive delivery system of claim 1 wherein the one or more apertures comprise a slit.

9. The adhesive delivery system of claim 1 wherein the one or more apertures follow a straight line.

10. The adhesive delivery system of claim 1 wherein the delivery conduit comprises a plurality of apertures following a non-planar scalloped shape.

11. The adhesive delivery system of claim 1 wherein the delivery conduit comprises a plurality of apertures following a generally circular shape.

12. The adhesive delivery system of claim 1 further comprising an adhesive reservoir connected to the central lumen.

13. The adhesive delivery system of claim 12 further comprising a valve to control the release of adhesive from the reservoir to the delivery conduit.

14. The adhesive delivery system of claim 12 further comprising a syringe controlling flow from the adhesive reservoir to the delivery conduit.

15. The adhesive delivery system of claim 12 wherein the adhesive reservoir comprises a medical adhesive selected from the group consisting of a fibrin glue, a urethane polymer and a cyanoacrylate compound.

16. The adhesive delivery system of claim 1 further comprising a prosthesis attached to the delivery conduit.

17. The adhesive delivery system of claim 16 wherein an adhesive reservoir is incorporated into the prosthesis.

18. The adhesive delivery system of claim 1 further comprising a tubular prosthesis attached to the delivery conduit.

19. The adhesive delivery system of claim 1 further comprising a heart valve prosthesis attached to the delivery conduit.

20. The adhesive delivery system of claim 19 wherein the heart valve prosthesis is an aortic heart valve prosthesis.

21. The adhesive delivery system of claim 19 wherein the aortic heart valve prosthesis is stentless and wherein the delivery conduit comprises a plurality of apertures-following a scalloped shape extending near the outflow edge of the prosthesis.

22. The adhesive delivery system of claim 1 further comprising an annuloplasty ring attached to the delivery conduit.

23. The adhesive delivery system of claim 1 wherein the delivery conduit is releasably attached to an adhesive reservoir.

24. A prosthesis comprising an attachment surface and an adhesive delivery conduit associated with the attachment surface, the adhesive delivery conduit having at least one aperture and a central lumen connected to the aperture.

25. The prosthesis of claim 24 wherein the delivery conduit is curved.

26. The prosthesis of claim 24 wherein the prosthesis comprises a heart valve prosthesis.

27. The prosthesis of claim 24 wherein the adhesive delivery conduit is connected to an adhesive reservoir.

28. The prosthesis of claim 24 wherein the delivery conduit is attached to the prosthesis with suture, clips or hooks.

29. The prosthesis of claim 24 wherein the prosthesis comprises a stentless aortic heart valve prosthesis having a scalloped outflow edge and wherein the delivery conduit follows a closed curve near the scalloped outflow edge.

30. A method for preparing a prosthesis for implantation, the prosthesis comprising an attachment surface, the method comprising placing a delivery conduit with at least one aperture along the attachment surface.

31. The method of claim 30 wherein the prosthesis comprises a heart valve prosthesis.

32. The method of claim 30 wherein the delivery conduit comprises a perforated section following a closed curve.

33. The method of claim 30 wherein the placing of the delivery conduit comprises attachment of suture, hooks or clips around the delivery conduit.

34. The method of claim 30 wherein the delivery conduit is attached to an adhesive reservoir with a channel connecting the conduit and the adhesive reservoir and wherein flow control component controls the release of adhesive from the reservoir to the delivery conduit.

35. The method of claim 34 wherein the adhesive flow from the delivery conduit follows a curve with a continuous pattern.

36. The method of claim 35 wherein the adhesive flow from the delivery conduit follows a curve with an intermittent pattern.

37. A method for the implantation of a prosthesis comprising delivering a pattern of a composition selected from the group consisting of a medical adhesive, a medical adhesive component and treatment compound, through a delivery conduit comprising at least one aperture.

38. The method of claim 37 wherein the delivery conduit delivers adhesive along a curve.

39. The method of claim 37 wherein adhesive components from two adhesive reservoirs are mixed prior to the delivery through the aperture.

40. The method of claim 37 wherein the composition comprises an adhesive component and wherein another adhesive component is applied through a second delivery conduit with at least one aperture.

41. A method of making a medical device, the method comprising applying an adhesive through a delivery conduit and forming an adhesive bond with the adhesive connecting to prosthesis components.

42. An adhesive delivery system comprising an adhesive delivery conduit comprising a resorbable polymer.

43. The adhesive delivery system of claim 42 further comprising a metal support associated with the adhesive delivery conduit.

44. The adhesive delivery system of claim 42 wherein the resorbable polymer comprises D-polylactic acid, L-polylactic acid, poly(glycolic acid), and copolymers of at least two of L-lactic acid, D-lactic acid and glycolic acid.

45. The adhesive delivery system of claim 42 wherein the adhesive delivery conduit has one or more apertures following a pattern.

46. The adhesive delivery system of claim 45 wherein the one or more apertures follow a curve.

47. The adhesive delivery system of claim 45 wherein the one or more apertures comprise a plurality of apertures.

48. A prosthesis comprising an adhesive delivery system of claim 42.

49. The prosthesis of claim 48 comprising a heart valve prosthesis.

50. The prosthesis of claim 49 wherein the heart valve prosthesis is a stentless aortic heart valve prosthesis.

51. An adhesive delivery system comprising an adhesive delivery conduit having a plurality of microtentacles/microtubules extending from the surface of the adhesive delivery conduit.

52. The adhesive delivery conduit of claim 51 wherein the microtentacles/microtubules are fenestrated.

53. The adhesive delivery conduit of claim 51 wherein the microtentacles/microtubules are non-fenestrated.

54. The adhesive delivery conduit of claim 51 wherein the adhesive delivery conduit comprises one or more apertures following a pattern.

55. The adhesive delivery conduit of claim 54 wherein the one or more apertures follow a curve.
